(19) United States
(12) Patent Application Publication
(10) Pub. No.: US 2015/0209397 A1
(43) Pub. Date: Jul. 30, 2015

(54) METHODS AND MATERIALS FOR REDUCING RETICULOENDOTHELIAL SYSTEM CLEARANCE OF PARTICLES FROM A SUBJECT

(71) Applicant: CARNEGIE MELLON UNIVERSITY, Pittsburgh, PA (US)

(72) Inventors: Chien Ho, Pittsburgh, PA (US); Li Liu, Pittsburgh, PA (US); Yijen Wu, Pittsburgh, PA (US); T. Kevin Hitchens, Pittsburgh, PA (US); Qing Ye, Pittsburgh, PA (US)

(21) Appl. No.: 14/426,569

(22) PCT Filed: Sep. 6, 2013
(86) PCT No.: PCT/US13/58587

§ 371 Date: Mar. 6, 2015

(30) Foreign Application Priority Data
Sep. 7, 2012 (US) 61743610

(51) Int. Cl. A61K 36/48; A61K 49/18; A61K 31/047; A61K 9/107; A61K 31/683

(52) U.S. Cl. CPC A61K 36/48; A61K 9/107; A61K 31/683; A61K 31/047; A61K 49/1833; A61K 49/1818

(57) ABSTRACT

Methods and materials for reducing clearance of particles such as nanoparticles or micron-sized particles from a subject are described. The methods include preadministering a fat emulsion before administering the particles.

### CROSS-REFERENCE TO RELATED APPLICATIONS

This application claims priority to U.S. Application Ser. No. 61/743,610, filed Sep. 7, 2012, the disclosure of which is incorporated by reference it its entirety.

### STATEMENT REGARDING FEDERALLY SPONSORED RESEARCH OR DEVELOPMENT

This invention was made with government support under the National Institutes of Health R01HL-081349 and P41EB-001977. The government has certain rights in this invention.

### TECHNICAL FIELD

This invention generally relates to methods for reducing reticuloendothelial system (RES) clearance of particles from a subject, and more particularly to pre-administering a fat emulsion to the subject to reduce clearance of particles administered to the subject.

### BACKGROUND

Nanoparticles are increasingly developed for use in medicine as theranostic or diagnostic agents, for targeted drug delivery, as well as for in vivo cellular/molecular imaging applications. For example, iron-oxide nanoparticles are useful for various biomedical applications including as contrast agents in magnetic resonance imaging (MRI), targeted drug delivery, and therapy. However, in-vivo uses of iron-oxide and other nanoparticles can be hampered by their rapid clearance from circulation by the reticuloendothelial system (RES). See, Neuberger, et al., J. Magn. Magn. Mater., 293:483-496 (2005). Studies have reported that the majority of injected iron-oxide particles are taken up by the RES, in particular by the liver Kupffer cells. See, e.g., Chouly, et al., J Microencapsul., 13:245-255 (1996); Okon, et al., Lab Invest., 71:895-903 (1994). In order to improve the usefulness of nanoparticles for imaging and drug delivery applications, it is beneficial to minimize the clearance by the RES.

Strategies that decrease the RES clearance and prolong the circulation residence time of nanoparticles can improve in-vivo targeting efficiency and lower the required effective dose of nanoparticles. Studies have been conducted to optimize the size, charge, surface property, and composition of iron-oxide particles to increase the circulation lifetime for improved cellular imaging. For example, larger-sized particles are eliminated from bloodstream faster than smaller-sized particles. See, Neuberger, et al., 2005 supra. Neutral and zwitterionic nanoparticles exhibit longer circulation time than negatively and positively charged nanoparticles. Arvizo, et al., PLoS One, 6:e24374 (2011). In addition, a surface coating of polyethylene glycol (PEG) and modifications of nanoparticles with liposomes can reduce uptake by the RES, thus prolonging their circulation in the bloodstream. See, e.g, Maesaki, Curr Pharm Des., 8:433-440 (2002); and Romberg, et al., Pharm Res., 25:55-71 (2008). Modifying the particle characteristics is effective in reducing RES clearance; however, it is not always or achievable, or desirable. Thus, there is a need for a more broadly applicable strategy to reduce RES clearance.

### SUMMARY

The present document is based, at least in part, on methods for reducing RES clearance of particles in a subject (e.g., a human patient) by pretreating with a fat emulsion. The methods described herein can, for example, reduce liver uptake of particles and/or increase the blood half-life of particles (e.g., nano or micron sized particles) in the subject. For example, as described herein, administering a fat emulsion to a subject before administering particles to the subject can result in an approximately 50% decrease in liver uptake and about a 3-fold increase in blood half-life of nano- or micron-sized particles (e.g., ultrasmall superparamagnetic iron oxide particles (USPIO) or micron sized superparamagnetic iron oxide particles (MPIO)), resulting in a 2- to 5-fold increase in the labeling efficiency of monocytes/macrophages in the peripheral blood. The results described herein demonstrate that pre-administering a fat emulsion to the subject can affect the pharmacokinetics of nano- and micron-sized particles, thus improving drug targeting and imaging applications of these particles for targets other than the liver.

In one aspect, this document features a method of reducing RES clearance of particles from a subject. The method comprises administering a fat emulsion to the subject and administering particles to the subject, wherein the fat emulsion is administered between 0 hours and 24 hours before the particles are administered to the subject.

In another aspect, this document features a method of increasing labeling efficiency of cells in a subject. The method comprises administering a fat emulsion to the subject and administering particles to the subject, the particles comprising an imaging agent, wherein the fat emulsion is administered between 0.2 hours and 24 hours before the particles are administered.

In any of the methods described herein, the fat emulsion can be administered between 0.3 and 4 hours, 0.3 and 3 hours, 0.5 and 1.5 hours, 4 and 10 hours, 10 and 24 hours, or 10 and 15 hours before the particles are administered. The fat emulsion can be administered about 1 hour before the particles are administered. The fat emulsion and the particles can be co-administered (e.g., in separate dosage forms). The fat emulsion can be administered intravenously to the subject. The methods described herein can increase targeting of the particles in the subject.

In any of the methods described herein, a reduced dosage of the particles can be administered to the subject relative to the dosage administered to a corresponding subject without preadministration of the fat emulsion.

In any of the methods described herein, the dosage of particles administered to the subject can be 20% to 40% of the dosage administered to a corresponding subject without preadministration of the fat emulsion.

In any of the methods described herein, the method further can include tracking cells comprising the particles in the subject using an imaging method (e.g., magnetic resonance imaging).

In any of the methods described herein, the particles can be superparamagnetic iron-oxide particles.

In any of the methods described herein, the particles can be micron sized particles or nanoparticles.

In any of the methods described herein, the particles can comprise an imaging agent, a therapeutic agent, a dye such as a fluorescent dye, a targeting agent such as an antibody, an antibody fragment, a protein, a peptide, an oligonucleotide, or a small molecule, and combinations thereof.

In any of the methods described herein, the method can further include assessing function of a biological sample from the subject using an imaging method.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and the drawings, and from the claims. The word “comprising” in the claims may be replaced by “consisting essentially of” or with “consisting of,” according to standard practice in patent law.

### DESCRIPTION OF DRAWINGS

FIGS. 1A and 1B are bar graphs of the changes in iron levels, as measured by inductively coupled plasma-mass spectrometry (ICP-MS), in different tissues (liver, spleen, kidney, lung, and heart) at 48 hrs following administration of USPIO-NH₂-FITC particles (FIG. 1A) or MPIO particles (FIG. 1B) either without Intralipid® pretreatment or one hour after Intralipid® pretreatment. Tissues from male BN rats, treated with PBS, were used as controls. *p<0.01 compared with iron-oxide particles administered without Intraplipid® pretreatment.

FIGS. 2A-2B depict photomicrographs of histological sections showing the changes in iron levels in liver samples at 48 hr following infusion of USPIO-NH₂-FITC particle either without Intralipid® pretreatment (2A) or one hour after Intralipid® pretreatment (2B). FIGS. 2C and 2D depict photomicrographs of histological sections showing the changes in iron levels in liver samples at 48 hr following infusion of MPIO particles either without Intralipid® pretreatment (2C) or one hour after Intralipid® pretreatment (2D). Sections were stained with Perl's Prussian blue and observed under a light microscope at 200× magnification.

FIGS. 3A and 3B depict line graphs of the changes in blood clearance of USPIO-NH₂-FITC particles (FIG. 3A) or MPIO particles (FIG. 3B) either without Intralipid® pretreatment (filled circles) or one hour after Intralipid® pretreatment (filled triangles).

FIG. 4A depicts flow cytometry dot plots of the kinetics of blood monocytes labeled by administration of USPIO-NH₂-FITC particles either without Intralipid® pretreatment or one hour after Intralipid® pretreatment. FIG. 4B is a summary of the flow cytometry dot plots of FIG. 4A.

FIG. 4C depicts flow cytometry dot plots of the kinetics of blood monocytes labeled by administration of MPIO particles either without Intralipid® pretreatment or one hour after Intralipid® pretreatment. FIG. 4D is a summary of the flow cytometry dot plots of FIG. 4C.

FIGS. 5A and 5B depict bar graphs of the changes in the iron levels, as measured by the transverse relaxation rate (R₂), in different tissues (liver, spleen, kidney, lung, and heart) at 48 hrs following administration of USPIO-NH₂-FITC particles (FIG. 5A) or MPIO particles (FIG. 5B) either without Intralipid® pretreatment or one hour after Intralipid® pretreatment. The tissues from male BN rats, treated with PBS, were used as controls, *p<0.01 and **p<0.05 compared with the iron-oxide particles administered without Intraplipid® pretreatment, respectively.

### DETAILED DESCRIPTION

This document provides methods and materials for reducing RES clearance of particles in a subject (e.g., a mammalian subject such as a human or other non-human primate, a rat, guinea pig, mouse, or a farm animal such as a pig, sheep, goat, horse, or cow). The methods and materials described herein include administering a fat emulsion to a subject up to about 24 hours before the particles are administered to, for example, reduce the RES clearance of particles in the subject, modulate the pharmacokinetics and prolong the circulation time of the particles, reduce the dosage of particles administered to the subject, temporarily inhibit Kuppfer cell activity, and/or increase the labelling efficiency of immune cells such as monocytes/macrophages for in vivo imaging and/or cell tracking

In the methods described herein, an amount of fat emulsion effective to reduce clearance of the particles is administered to the mammal. As used herein, the term “effective amount” refers to an amount (or amounts) of a fat emulsion that reduces clearance of particles without inducing significant toxicity to the host. For example, an effective amount of fat emulsion can range from, for example, about 0.1 g of fat emulsion/kg of body weight (mg/kg) to about 4 g/kg (e.g., 0.5 to 1.0, 0.75 to 1.5, 1 to 3, 1 to 4, 1.5 to 3, 1.5 to 4, 2 to 4, 1, 1.5, 2, 2.5, 3, 3.5, or 4 mg/kg). An effective amount of fat emulsion as well as frequency and duration of administration can be determined by a physician, taking into account various factors that can modify the action of drugs such as overall health status, body weight, sex, diet, time and route of administration, other medications, and any other relevant clinical factors.

While in some embodiments, a single pretreatment (e.g., a single intravenous administration) of fat emulsion is sufficient to reduce clearance of particles from the subject, in some embodiments, pretreatment can comprise multiple administrations of the fat emulsion at various intervals and over different periods of time as required.

Typically, the fat emulsion is administered between 0 hours and 24 hours before the administration of the particles. For example, the fat emulsion can be administered between 0.1 and 24 hours, 0.1 and 20 hours, 0.1 and 16 hours, 0.1 and 15 hours, 0.1 and 10 hours, 0.1 and 5 hours, 0.1 and 2.5 hours, 0.2 and 24 hours, 0.2 and 20 hours, 0.2 and 15 hours, 0.2 and 10 hours, 0.2 and 5 hours, 0.2 and 2.5 hours, 0.3 and 4.5 hours, 0.3 and 4 hours, 0.3 and 3 hours, 0.5 and 24 hours, 0.5 and 20 hours, 0.5 and 15 hours, 0.5 and 10 hours, 0.5 and 5 hours, 0.5 and 3.5 hours, 0.5 and 2 hours, 0.5 and 1.5, 1 and 24 hours, 1 and 15 hours, 1 and 10 hours, 1 and 5 hours, 1 and 3 hours, 1 and 2.5 hours, 1 and 1.5 hours, 2 and 24 hours, 2 and 20 hours, 2 and 15 hours, 2 and 10 hours, 2 and 5 hours, 5 and 24 hours, 5 and 20 hours, 5 and 15 hours, 5 and 10 hours, 10 and 24 hours, 10 and 20 hours, or 10 and 15 hours before the particles are administered. In some embodiments, the fat emulsion is administered about 1 hour before the particles are administered. In some embodiments, the fat emulsion and particles are co-administered.

In embodiments in which the fat emulsion is administered multiple times (e.g., two, three, four, or more times), at least one of the administrations is between 0 hours and 5 hours before the administration of the particles. In some embodiments, multiple administrations of the fat emulsion occur between 0 hours and 24 hours before the administration of the particles to the subject.

The fat emulsion can be formulated for administration by any route, including, without limitation, oral or parenteral routes of administration such as intravenous, intramuscular, intraperitoneal, subcutaneous, intrathecal, intraarterial, or nasal. Typically, the fat emulsion is administered intravenously.

In some embodiments, the fat emulsion comprises one or more of soybean oil or other vegetable oil, fish oil, phospholipids (e.g., egg yolk phospholipids), and glycerol or other pharmaceutically acceptable carrier, or combinations and subcombinations of the foregoing. Intralipid® is a particularly useful fat emulsion. Intralipid® was approved by U.S. FDA in 1972 as a source of parenteral nutrition for patients. Intralipid® 20% is composed of 20% soybean oil, 1.2% egg-yolk phospholipids, and 2.25% glycerol. Other Intralipid® formulations include Intralipid® 10% and Intralipid® 30%. Kupffer cells in the liver play an important role in the uptake and metabolism of Intralipid. See, for example, Vilaro and Llobera, J Nutr., 118:932-940 (1988). Intralipid infusion has been reported to inhibit RES function by possibly inhibiting peritoneal clearance and impairing the phagocytic activity of peritoneal macrophages. See, for example, Nugent, J Leukoc Biol., 36:123-132 (1984).

The methods described herein further comprise administering particles to the subject from about 0 to 24 hours after the fat emulsion. For example, the particles can be administered between 0 between 0.1 and 24 hours, 0.1 and 20 hours, 0.1 and 16 hours, 0.1 and 15 hours, 0.1 and 10 hours, 0.1 and 5 hours, 0.1 and 2.5 hours, 0.2 and 24 hours, 0.2 and 20 hours, 0.2 and 15 hours, 0.2 and 10 hours, 0.2 and 5 hours, 0.2 and 2.5 hours, 0.3 and 4.5 hours, 0.3 and 4 hours, 0.3 and 3 hours, 0.5 and 24 hours, 0.5 and 20 hours, 0.5 and 15 hours, 0.5 and 10 hours, 0.5 and 5 hours, 0.5 and 3.5 hours, 0.5 and 2 hours, 0.5 and 1.5, 1 and 24 hours, 1 and 15 hours, 1 and 10 hours, 1 and 5 hours, 1 and 3 hours, 1 and 2.5 hours, 1 and 1.5 hours, 2 and 24 hours, 2 and 20 hours, 2 and 15 hours, 2 and 10 hours, 2 and 5 hours, 5 and 24 hours, 5 and 20 hours, 5 and 15 hours, 5 and 10 hours, 10 and 24 hours, 10 and 20 hours, or 10 and 15 hours after the fat emulsion is administered to the subject. In some embodiments, the particles are administered about 1 hour after the fat emulsion is administered. In some embodiments, the fat emulsion and particles are co-administered.

While in some embodiments, a single administration (e.g., a single intravenous administration) of particles is sufficient, in some embodiments, multiple administrations of the particles at various intervals and over different periods of time are required. In embodiments in which the particles are administered multiple times (e.g., two, three, four, or more times), at least one of the administrations is between 0 hours and 24 hours after the administration of the fat emulsion. In some embodiments, multiple administrations of the particle occur between 0 hours and 24 hours after the administration of the fat emulsion to the subject.

Methods described herein can be used with particles of any size (e.g., nano- or micron sized) and of any material suitable for administration to the subject. For example, the particles can be silica oxide particles, metal oxide particles such as zinc, aluminum, or iron oxide particles, superparamagnetic iron platinum particles, gadolinium particles, manganese particles, gold particles, silver particles, lipid particles, polyacrylic acid particles, or polymer particles such as poly(lactide-coglycolic acid) (PLGA) polymers. In some embodiments, the particles are iron oxide particles such as superparamagnetic iron oxide particles (e.g., ultrasmall superparamagnetic iron oxide particles (USPIO) or micron sized superparamagnetic iron oxide particles (MPIO)). The particles can be neutral, zwitterionic, negatively, or positively charged. In addition, in some embodiments, the surfaces of the particles are coated, for example, with a dextran, a dendrimer (e.g., a carboxylated dendrimer), styrene/divinyl benzene, or vinyl polymer, or modified, for example, with a liposome, or polyethylene glycol (PEG) or other polyether. See, e.g., Bulte and Kraitchman, NMR Biomed., 17:484-499 (2004).

In some embodiments, the surface of the particles can comprise reactive functional groups such as thiols, chloromethyl, bromomethyl, hydroxyls, amines, carboxylic acid or activated ester, vinylsulfonyls, aldehydes, epoxies, hydrazides, succinimidyl esters, maleimides, α-halo carbonyl moieties (such as iodoacetyls), isocyanates, isothiocyanates, 4-fluoro-5-nitro-benzoate, and aziridines. For example, the surface of the particle can comprise a reactive functional group such as a thiol, a carboxylic acid, an amine, 4-fluoro-5-nitro-benzoate, or a carboxylic acid activated ester. Such reactive functional groups are useful for linking an imaging agent, a therapeutic, targeting agent, or other molecule to the particle.

In some embodiments, the particles further comprise one or more imaging agents such as an agent used in photoacoustic imaging, fluorescence imaging, positron emission tomography (PET), computer assisted tomography (CAT), single-photon emission computed tomography (SPECT), optical imaging, or magnetic resonance imaging (MRI). For example, the imaging agent can be a radioisotope of carbon, iodine, nitrogen, oxygen, fluorine, or rubidium, a fluorescent dye such as indocyanine green, quantum dot, or a near-infrared fluorescent dye, and/or a fluorescent quencher.

In some embodiments, the particles further can comprise a therapeutic agent such as a chemotherapeutic (e.g., an alkylating agent, anti-metabolite, anti-microtubule or microtubule stabilizing agent, topoisomerase inhibitor, or cytotoxic antibiotic) (see, for example, Hu, et al., Ther Deliv. 1(2):323-34 (2010)), an antibiotic (see, for example, Zhang, et al., Current Medicinal Chemistry, 17:585-594 (2010)), or anti-inflammatory or anti-angiogenic agent (see, for example, Laroui, et al., Methods Enzymol., 509:101-25 (2012)), or a targeting agent such as an antibody (e.g., a monoclonal antibody such as a monoclonal antibody having binding affinity for epidermal growth factor, human epidermal growth factor receptor 2, vascular endothelial growth factor, interleukin-2 receptor alpha, interleukin-1 beta, interleukin-6 receptor, CD20, CD30, or CD52) (see, for example, Barua, et al., Proc Natl Acad Sci USA, 110(9):3270-5 (2013)), an antibody fragment (e.g., a single chain Fv antibody fragment, Fab fragments, or F(ab)₂ fragment) (see, for example, Yang, et al., Small, 5:235-43 (2009)), a bispecific antibody, a protein (see, for example, Hu, et al., Angew Chem Int Ed Engl, 46:5111-5114 (2007)), a peptide (e.g., polylysine) (see, for example, Rodriguez, et al., Science, 339: 971-975 (2013)), an oligonucleotide (see, for example, Rosi, et al., Science, 312:1027-1030 (2006)), a small molecule (see, for example, Zhao, et al., Nat. Mater., 8:979-985 (2009)), or other affinity ligand.

In some embodiments, the particles can comprise one or more of an imaging agent, a therapeutic, or a targeting agent. For example, in some embodiments, the particles comprise a therapeutic and a targeting agent, an imaging agent and a targeting agent, or a therapeutic agent, targeting agent, and an imaging agent.

The particles can be formulated for administration by any route, including, without limitation, oral or parenteral routes of administration such as intravenous, intramuscular, intraperitoneal, subcutaneous, intrathecal, intraarterial, or nasal. Typically, the particles are administered intravenously.

Formulations of particles may comprise a pharmaceutically acceptable carrier. As used herein the term “pharmaceutically acceptable carrier” comprises solvents, dispersion media, coatings, isotonic and absorption delaying agents, and the like, compatible with pharmaceutical administration. For example, particles can be formulated with one or more pharmaceutically acceptable carriers or excipients suitable for the particular route of administration, including sterile aqueous or non aqueous carriers. Aqueous carriers comprise, without limitation, water, alcohol, saline, and buffered solutions. Examples of non aqueous carriers comprise, without limitation, propylene glycol, polyethylene glycol, vegetable oils, and injectable organic esters. Preservatives, flavorings, sugars, polyalcohols (e.g., mannitol or sorbitol), and other additives such as antimicrobials, antioxidants, chelating agents, inert gases, and the like also may be present. One or more agents that delay absorption such as aluminum monostearate or gelatin can be included to prolong absorption of the injectable compositions.

For oral administration, tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). Tablets can be coated by methods known in the art. Preparations for oral administration also can be formulated to give controlled release of the compound.

Nasal preparations can be presented in a liquid form or as a dry product. Nebulized aqueous suspensions or solutions can comprise carriers or excipients to adjust pH and/or tonicity.

The dosage of particles to be administered can depend on the desired use of the particles. For example, the dosage required for an imaging method (e.g., MRI) may vary from that required for delivering a therapeutic agent or targeting agent. Typical dosages of USPIO for imaging methods can range from 4.5 to 15 mg/kg. Typical dosages of MPIO for imaging methods can range from 4.5 to 18 mg/kg. The methods described herein can result in using a reduced dosage relative to the dosage that would be administered to a corresponding subject without preadministration of the fat emulsion. For example, using the methods described herein, the dosage of particles administered to the subject can be reduced about 5% to about 50% (e.g., about 10% to about 40%, 20% to about 40%, 20%, 25%, 30%, 35%, 40%, or 45%) of that typically administered to a subject.

In some embodiments, the methods described herein further can comprise assessing the subject using an imaging method (e.g., MRI) to, for example, label or track cells, determine location of the particles, detect structural abnormalities within the subject, distinguish between pathologic and normal tissue, or diagnose the subject as having a particular disease or disorder. For example, phagocytic immune cells (e.g., monocytes and macrophages) can be labeled in vivo by iron-oxide particles. Thus, an imaging method such as MRI can be used to track the labeled cells (i.e., comprising the particles) in the subject. See, for example, Ye, et al., Kidney Int. 61(3):1124-35 (2002); Wu, et al., Proc. Natl. Acad. Sci. USA, 103:1852-1857 (2006); and Wu, et al., JACC Cardiovasc Imaging, 2:731-741 (2009).

In some embodiments, the methods described herein further can comprise monitoring the subject to determine if, for example, treatment with the therapeutic agent results in an improvement in the condition for which the subject is being treated.

In some embodiments, a biological sample can be obtained from the subject (e.g., human) and assessed using an imaging method. For example, a biological sample such as blood (including whole blood, plasma, and serum), urine, or tissue can be obtained from the subject and assessed for function using an imaging technique.

The invention will be further described in the following examples, which do not limit the scope of the invention described in the claims.

### EXAMPLES

### Example 1—Methods and Materials

Animal Experiments—Male inbred Brown Norway (e.g., BN; RT1ⁿ) rats, purchased from Harlan (Indianapolis, IN) and having body weights between 250 and 280 g, were used in the experiments described herein. All experiments involving animal subjects were approved by the Institutional Animal Care and Use Committee of Carnegie Mellon University. Animal care was provided in accordance with the Guide for the Care and Use of Laboratory Animals.

Biodistribution and iron levels in different tissues—The wet weight of each tissue sample was recorded and 1 mL of tissue homogenate was lyophilized in a test tube for 72 hr. One mL of 6 N HCl was added to each dry tissue sample and the samples were heated overnight in an incubator set at 55° C. The samples were centrifuged at 1,000 rpm for 15 min and the supernatant collected in a separate test tube. Suitable dilutions of the sample solution were prepared to reach the final concentration in the range of 0.02 to 1 part per million (ppm) with respect to iron. Samples were analyzed for iron concentrations by inductively ICP-MS (NexION 300X, PerkinElmer Inc., Waltham, Mass.). Suitable dilutions of standard solutions purchased from CPI International (Santa Rosa, Calif.) were prepared and a standard curve in the range of 0.02 to 1 ppm Fe was prepared. ⁵⁷Fe isotope counts were used to determine the Fe content.

The iron levels in different tissues were also determined by the transverse relaxation rate R₂. The R₂ values of tissue samples were measured at 20 MHz using a Bruker Minispec mq20 NMR Analyzer.

Blood half-life—The R₂ values of blood samples collected at different time points were measured using, in one example, a Bruker Minispec mq20 NMR analyzer. Blood clearance half-life was determined by fitting the R₂ values to a mono-exponential decay using, in one example, KaleidaGraph 4.1 (e.g., Synergy Software, Reading, Pa.).

Flow cytometry—Red blood cells in the blood samples were lysed with ACK lysing buffer. Mouse anti-rat ED1: Alexa Fluor 647 antibody (e.g., AbD SeroTec, Oxford, UK) was used to label monocytes and macrophages. In one example, BUF09 (AbD SeroTec) was used as a permeabilization reagent for ED1 detection. In one example, flow cytometry was performed on a FACSVantage (e.g., Becton Dickinson, Franklin Lakes, N.J.). The data were processed with the use of, FlowJo software (e.g., TreeStar, Ashland, Oreg.).

Iron content of labeled-monocytes—Approximately 48 hr following injection of USPIO-NH₂-FITC particles and 20 min or 48 hr after MPIO injection, 7 mL of blood was collected from each rat. Blood monocytes were stained as described above. The USPIO-NH₂-FITC or MPIO-labeled monocytes were sorted with the use of flow cytometry and the numbers of cells are recorded. The collected cells were dissolved in 50 μL of 70% nitric acid. The solutions were then subjected to ICP-MS analysis.

Histological analysis—Histological examinations were performed by the Transplantation Pathology Laboratory of the University of Pittsburgh Medical Center (Pittsburgh, Pa.). Perl's Prussian blue staining was conducted for detecting the presence of iron.

Statistical analysis—The iron levels in different tissues with and without Intralipid pretreatment were analyzed by Student's t test. A probability value <0.05 was considered statistically significant.

### Example 2

### Decreased Uptake of Iron-Oxide Particles with Intralipid Pretreatment

Intralipid® 20.0% (e.g., Fresenius Kabi, Bad Homburg, Germany) was administered by intravenous injection into male BN rats at a dose of 2 g/kg (n=30). Phosphate-buffered-saline (PBS, Mediatech, Inc. Manassas, VA) was administered to male BN rats (n=30) as a control. Iron-oxide particles were injected intravenously into male BN rats one hour after administration of Intralipid®. Nano-sized USPIO-NH₂-FITC particles were injected at a dose of 4.5 mg Fe/kg body weight and MPIO particles (0.9 μm in size, Bangs Laboratories, Fishers, Ind.) were injected at a dose of 6 mg Fe/kg body weight. MPIO particles contain a magnetite core as well as a fluorescent dye (Dragon Green).

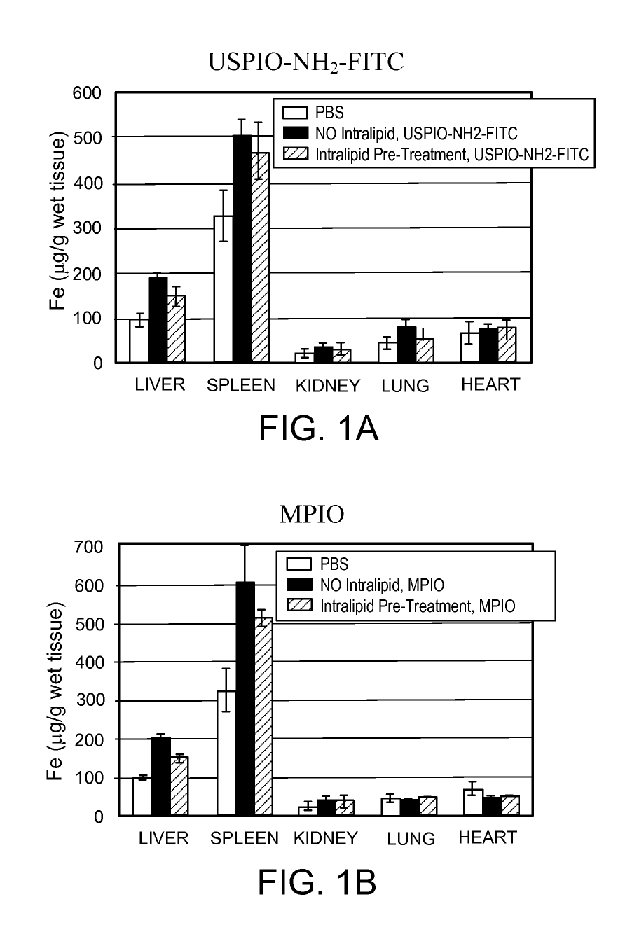

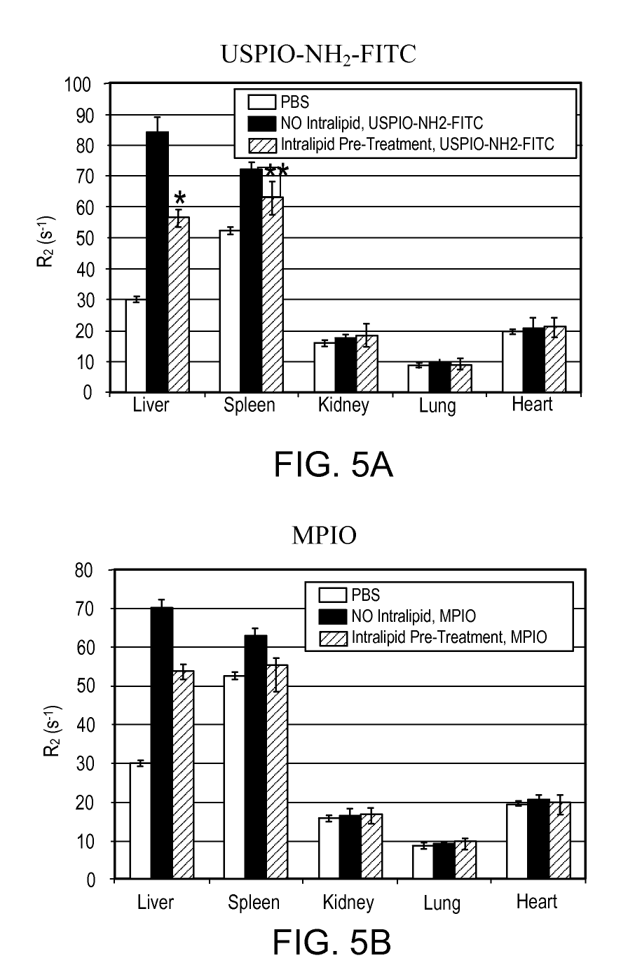

Blood samples were collected at different time points to determine the blood half-life of the particles and labeling efficiency of monocytes. Approximately 48-hr post injection of iron-oxide particles, various tissues (liver, spleen, kidney, lung, and heart) were collected for the iron-level determination and histological analysis. Iron concentration was either measured by ICP-MS (FIGS. 1A and 1B) or by R₂ values. Greater fractions of nano- or micron-sized iron-oxide particles were found in the liver and spleen, particularly the liver, than in the heart, kidney, or lung (see. FIGS. 1 and 5).

In the liver, the iron concentration increased from 101.0±8.5 (μg/g wet weight, control PBS injection) to 189.2±10.5 (μg/g wet weight) 48 hr after USPIO-NH₂-FITC, while in the spleen, the iron concentration increased from 329.5±55.2 (μg/g wet weight, control PBS injection) to 505.6±32.7 (μg/g wet weight) (FIG. 1A). This translates into an approximate 64.9% uptake in the liver and an approximate 8.7% uptake in the spleen of the injected iron.

Iron concentration also increased 48 hr after MPIO injection from 101.0±8.5 (μg/g wet weight) to 201.9±12.9 (μg/g wet weight) in the liver and from 329.5±55.2 (μg/g wet weight) to 603.0±89.5 (μg/g wet weight) in the spleen (FIG. 1B). Thus, approximately 55.6% and approximately 10.2% of the injected MPIO particles were localized in liver and spleen, respectively.

Upon pretreatment with a single dose of Intralipid® 20.0% (2 g/kg), the iron content in liver increased to 149.5±19.1 (μg/g wet weight) (FIG. 1A) and 155.9±10.5 (μg/g wet weight) (FIG. 1B) 48 hr after the injection of the USPIO-NH₂-FITC and MPIO particles, respectively. Thus, the uptake of the USPIO-NH₂-FITC particles in the liver significantly decreased by 45.1±6.5% and the uptake of MPIO particles significantly decreased by 49.2±5.9% compared to no pretreatment with Intralipid®. There was no significant change in uptake in the spleen following Intralipid® pre-treatment.

FIG. 5 shows the iron levels in different tissues as measured by the R₂ values. Upon pre-treatment with a single injection of Intralipid®, the liver uptake of both USPIO-NH₂-FITC and MPIO particles decreased significantly by 51.2±6.2% and 40.2±5.1%, respectively, relative to injection of the particles without Intralipid® pretreatment. In the spleen, a 45.9±11.5% reduction in the uptake of USPIO-NH₂-FITC particles and a 52.2±12.2% reduction in the uptake of MPIO particles also were observed upon pre-treatment with Intralipid®. There was variation in the iron concentrations and the R₂ values of the tissues collected from different animals, which could be due to the heterogeneous distribution of the iron oxide particles in the organ.

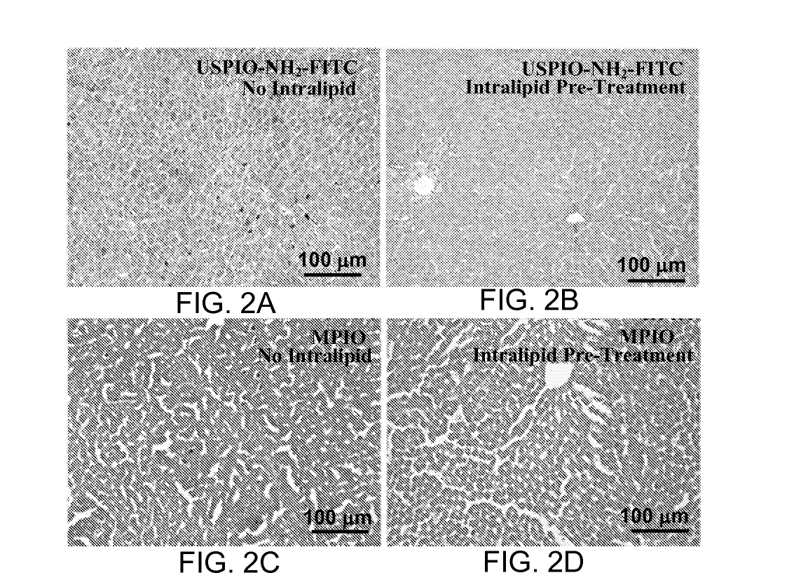

Histological examinations of the liver showed evidence of iron, as seen with Perl's Prussian blue staining, following the injection of USPIO-NH₂-FITC and MPIO particles (see FIGS. 2A and 2C). With Intralipid® pretreatment before the injection of the particles, the presence of iron stained Kupffer cells in the liver was dramatically decreased (see FIGS. 2B and 2D).

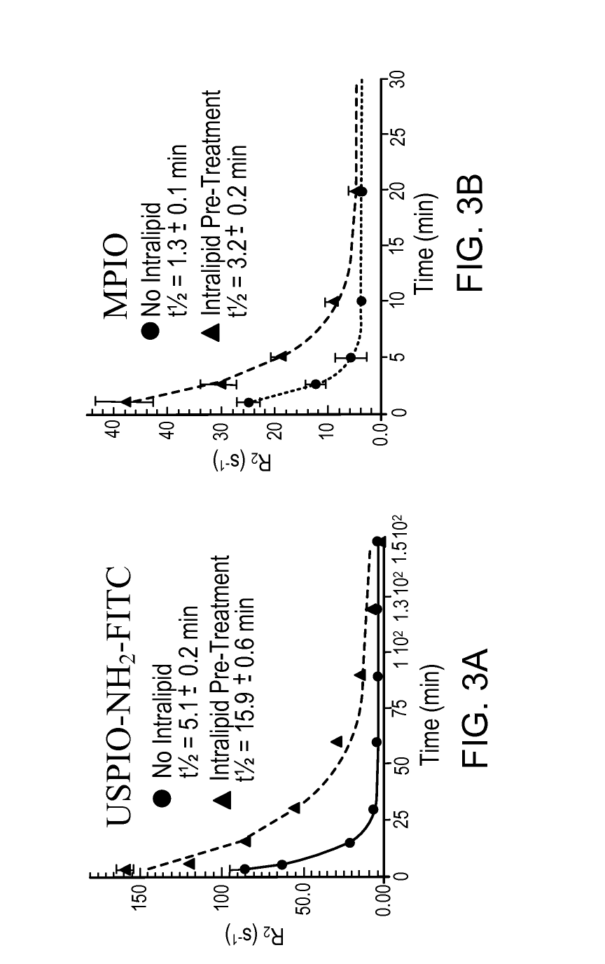

Pretreatment with Intralipid® produced a 3-fold increase in the blood half-life of USPIO-NH₂-FITC particles (FIG. 3A) and a 2.5-fold increase in the blood half-life of micron-sized iron-oxide particles (FIG. 3B), as determined by the changes in whole blood R₂ values following particle injection. A control experiment was conducted to confirm that Intralipid® 20% (diluted 15% v/v) had no effect on the R₂ value for aqueous solution of USPIO-NH₂-FITC and MPIO particles. The blood half-life of USPIO-NH₂-FITC particles was determined to be 5.1±0.2 min. With Intralipid® pre-treatment, the half-life increased to 15.9±0.6 min (FIG. 3A). The blood half-life of MPIO particles was 1.3±0.1 min. With Intralipid® pre-treatment, the blood half-life increased to 3.2±0.2 min (FIG. 3B).

### Example 3

### Decreased Uptake of Iron-Oxide Particles with Intralipid® Pretreatment

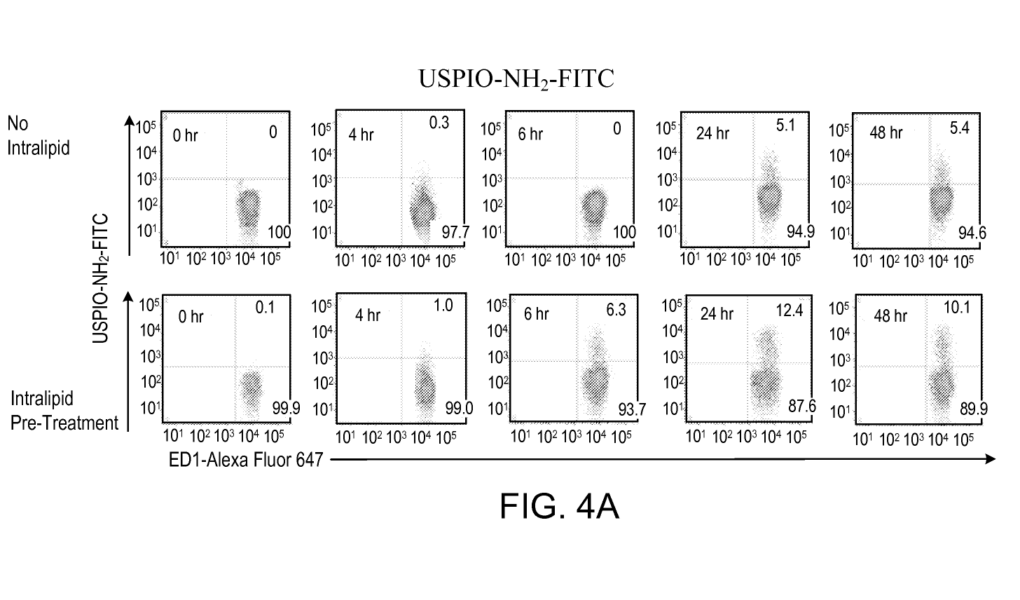

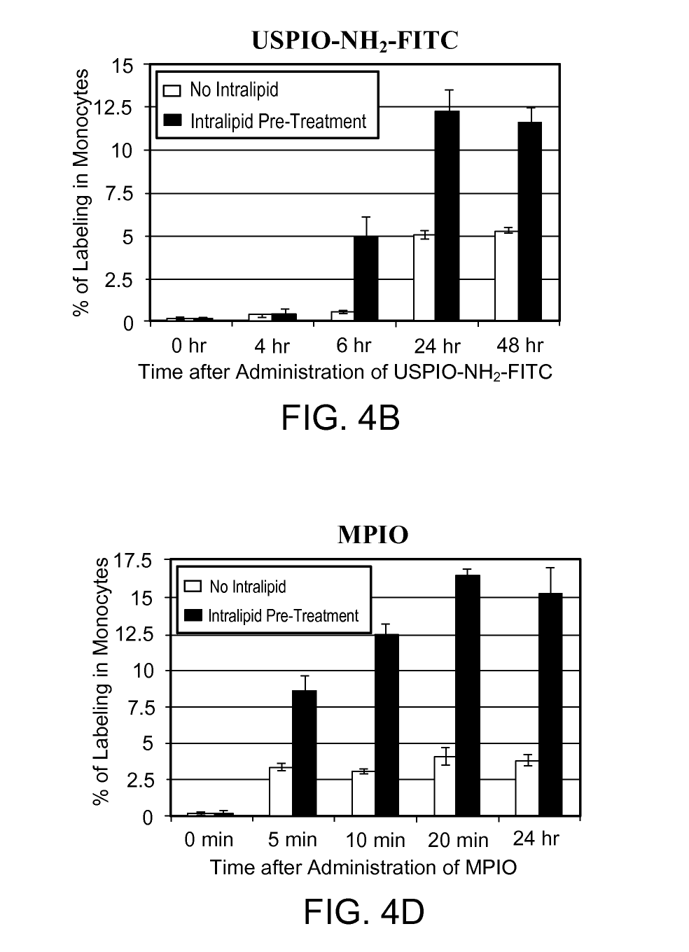

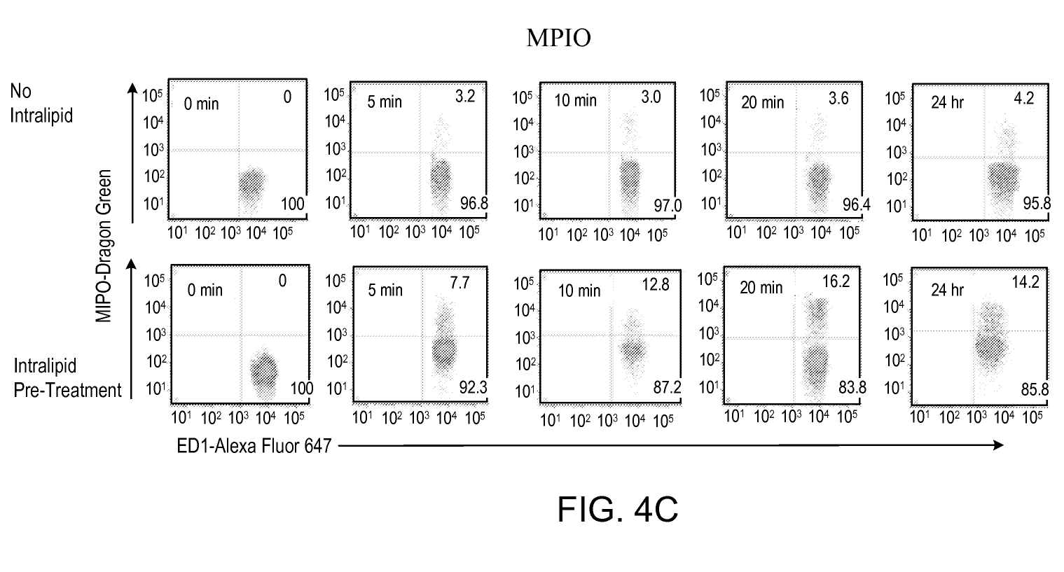

Phagocytic immune cells (e.g., monocytes and macrophages) can be labeled in vivo by iron-oxide particles. As described in this example, pretreatment with Intralipid® significantly enhances the labeling efficiency of blood monocytes/macrophages by nano- and micron-sized iron-oxide particles. FIG. 4 shows the kinetics of the appearance of USPIO-NH₂-FITC-labeled and MPIO-labeled monocytes in blood with and without Intralipid® pretreatment. USPIO-NH₂-FITC-labeled monocytes appeared in blood slowly after direct i.v. injection of the particles (FIG. 4A). Flow cytometry of blood samples does not detect USPIO-NH₂-FITC-labeled monocytes after 6 hr following particle injection (FIG. 4A, upper panel). Approximately 24-hr post USPIO-NH₂-FITC injection, 5.0±0.9% of the monocytes were labeled. After pre-treatment with Intralipid®, 5.0±1.1% and 12.2±1.3% of monocytes were labeled with USPIO-NH₂-FITC particles 6- and 24-hr post injection, respectively (see FIG. 4A, lower panel). FIG. 4B is a summary graph of the flow cytometry data.

In contrast to the nano-sized particles, MPIO-labeled monocytes were detected in blood shortly after i.v injection. The upper panel of FIG. 4C shows that 3.3±0.4% of blood monocytes were labeled with MPIO particles 5 min. after particle injection. This labeling efficiency does not change significantly when sampled at 24 hr. Upon pre-treatment with Intralipid®, 8.5±0.7% of blood monocytes were labeled 5 min after MPIO injection (FIG. 4C, lower panel). The labeling efficiency increases to 16.5±0.4% after 20 min and at 24 hr, the labeling efficiency was 15.2±1.9%. FIG. 4D is a summary graph of the flow cytometry data.

In one experiment, USPIO-NH₂-FITC- or MPIO-labeled-monocytes were sorted from white blood cells by flow cytometry. About 40,000 labeled-monocytes were harvested from the blood of rats without Intralipid® pre-treatment and ˜100,000 labeled-monocytes were harvested from Intralipid® pretreated rats. Changes in the iron concentrations in blood monocytes after iron-oxide particle injection following Intralipid® pretreatment are shown in Table 1.

TABLE 1
Effects of Intralipid ® on the iron concentrations in blood
monocytes after injection of iron-oxide particles.

| | | Duration | Iron |
|---|---|---|---|
| Pre- | Iron-Oxide | after Particle | Concentration (pg |
| Treatment | Particles | Injection | Fe/Monocyte) |
| PBS | PBS | 20 min | 0.04 ± 0.02 |
| PBS | USPIO-NH2-FITC | 48 hr | 0.28 ± 0.07 |
| Intralipid ® | USPIO-NH2-FITC | 48 hr | 0.22 ± 0.07 |
| PBS | MPIO | 20 min | 0.82 ± 0.12 |
| Intralipid ® | MPIO | 20 min | 2.60 ± 0.37 |
| PBS | MPIO | 48 hr | 1.21 ± 0.15 |
| Intralipid ® | MPIO | 48 hr | 2.92 ± 0.09 |

Blood monocytes from male BN rats, treated with PBS, served as controls. Pretreatment with Intralipid® increased the percentage of USPIO-NH₂-FITC-labeled monocytes (FIGS. 4A and 4B), without changing the intracellular iron concentration (˜0.2 to 0.3 pg Fe/monocyte). However, pretreatment with Intralipid® caused a three-fold increase in iron concentration of MPIO-labeled monocytes. After 20 min of MPIO injection, but without Intraplipid® pretreatment, the intracellular iron concentration in MPIO-labeled monocytes was found to be 0.82±0.12 pg Fe/monocyte, indicating that there are 1 to 2 MPIO particles in each monocyte (˜0.5 pg Fe/MPIO particle). Upon Intralipid® pre-treatment, the iron concentration increased to 2.60±0.37 pg Fe/monocyte, indicating that there were ˜5 MPIO particles in each MPIO-labeled monocyte. Approximately 48 hr following MPIO injection, the iron content in MPIO-labeled blood monocytes, without and with pretreatment with Intralipid®, increased to 1.21±0.15 pg Fe/monocyte and 2.92±0.09 pg Fe/monocyte, respectively.

In view of these experiments, it is shown that labeling of monocytes/macrophages by different iron-oxide particles may follow different pathways, and depending on the particles and/or other conditions taking place alternatively in circulation and/or in organs, such as the spleen and bone marrow.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing reticuloendothelial system clearance of particles from a subject, the method comprising (i) administering a fat emulsion to the subject and (ii) administering particles to the subject, wherein the fat emulsion is administered between 0 hours and 24 hours before the particles are administered to the subject.

2. The method of claim 1, wherein the fat emulsion is administered between 0.3 and 4 hours before the particles are administered.

3. The method of claim 1, wherein the fat emulsion is administered between 0.3 and 3 hours before the particles are administered.

4. The method of claim 1, wherein the fat emulsion is administered between 0.5 and 1.5 hours before the particles are administered.

5. The method of claim 1, wherein the fat emulsion is administered between 4 and 10 hours before the particles are administered.

6. The method of claim 1, wherein the fat emulsion is administered between 10 and 24 hours before the particles are administered.

7. The method of claim 1, wherein the fat emulsion is administered between 10 and 15 hours before the particles are administered.

8. The method of claim 1, wherein the fat emulsion is administered about 1 hour before the particles are administered.

9. The method of claim 1, wherein the fat emulsion and the particles are co-administered.

10. The method of claim 9, wherein the fat emulsion and the particles are co-administered in separate dosage forms.

11. The method of claim 1, wherein the fat emulsion is administered intravenously to the subject.

12. The method of claim 1, wherein the method increases targeting of the particles in the subject.

13. The method of claim 1, wherein a reduced dosage of the particles is administered to the subject relative to the dosage administered to a corresponding subject without preadministration of the fat emulsion.

14. The method of claim 1, wherein the dosage of particles administered to the subject is 20% to 40% of the dosage administered to a corresponding subject without preadministration of the fat emulsion.

15. The method of claim 1, the method further comprising tracking cells comprising said particles in the subject using an imaging method.

16. The method of claim 1, wherein the particles are superparamagnetic iron-oxide particles.

17. The method of claim 1, wherein the particles are micron sized particles.

18. The method of claim 1, wherein the particles are nanoparticles.

19. The method of claim 1, wherein the particles comprise an imaging agent.

20. The method of claim 1, wherein the particles comprise a therapeutic agent.

21. The method of claim 1, wherein the particles comprise a dye.

22. The method of claim 21, wherein the dye is fluorescent.

23. The method of claim 1, wherein the particles comprise a targeting agent.

24. The method of claim 23, wherein the targeting agent is selected from the group consisting of an antibody, an antibody fragment, a protein, a peptide, an oligonucleotide, and a small molecule.

25. A method of increasing labeling efficiency of cells in a subject, the method comprising (i) administering a fat emulsion to the subject and (ii) administering particles to the subject, the particles comprising an imaging agent, wherein the fat emulsion is administered between 0.2 hours and 24 hours before the particles are administered.

26. The method of claim 25, wherein the fat emulsion is administered between 0.3 and 4 hours before the particles are administered.

27. The method of claim 25, wherein the fat emulsion is administered between 0.3 and 3 hours before the particles are administered.

28. The method of claim 25, wherein the fat emulsion is administered between 0.5 and 1.5 hours before the particles are administered.

29. The method of claim 25, wherein the fat emulsion is administered between 4 and 10 hours before the particles are administered.

30. The method of claim 25, wherein the fat emulsion is administered between 10 and 24 hours before the particles are administered.

31. The method of claim 25, wherein the fat emulsion is administered between 10 and 15 hours before the particles are administered.

32. The method of claim 25, wherein the fat emulsion is administered about 1 hour before the particles are administered.

33. The method of claim 25, the method further comprising assessing function of a biological sample from the subject using an imaging method.

34. The method of claim 25, the method further comprising tracking cells comprising the particles in the subject using an imaging method.
